(19) United States
(12) Patent Application Publication
(10) Pub. No.: US 2024/0326029 A1
(43) Pub. Date: Oct. 3, 2024

(54) EPOXIDATION CATALYST AND PROCESS FOR ITS PREPARATION

(71) Applicant: BASF SE, Ludwigshafen am Rhein (DE)

(72) Inventors: Trees Maria DE BAERDEMAEKER, Ludwigshafen am Rhein (DE); Andrei-Nicolae PARVULESCU, Ludwigshafen am Rhein (DE); Joaquim Henrique TELES, Ludwigshafen am Rhein (DE); Jaroslaw Michael MORMUL, Ludwigshafen am Rhein (DE); Toshiyuki YOKOI, Tokyo (JP); Feiyu QIN, Yokohama (JP)

(21) Appl. No.: 18/711,894

(22) PCT Filed: Nov. 21, 2022
(86) PCT No.: PCT/EP2022/082643

§ 371 Date: May 21, 2024

(30) Foreign Application Priority Data
Nov. 22, 2021 (EP) 21209524.4

(51) Int. Cl. B01J 29/70; B01J 37/06; B01J 37/08; B01J 37/12; C01B 39/46

(52) U.S. Cl. CPC B01J 29/7049; B01J 37/06; B01J 37/08; B01J 37/12; C01B 39/46; C01P 2002/72; C01P 2002/82; C01P 2002/84; C01P 2004/03

(57) ABSTRACT

The present invention relates to a specific process for the preparation of a zeolitic material, wherein the framework structure of the zeolitic material comprises Si, O, and a tetravalent ele-ment M other than Si, wherein M is selected from the group consisting of Ti, Sn, Zr, Ge, and mixtures of two or more thereof. Further, the present invention relates to a zeolitic material obtainable or obtained by said process, the zeolitic material itself and its use. In addition thereto, the present invention relates to a molding comprising said zeolitic material.

### TECHNICAL FIELD

The present invention relates to a specific process for the preparation of a zeolitic material, wherein the framework structure of the zeolitic material comprises Si, O, and a tetravalent element M other than Si, wherein M is selected from the group consisting of Ti, Sn, Zr, Ge, and mixtures of two or more thereof. Further, the present invention relates to a zeolitic material obtainable or obtained by said process, the zeolitic material itself and its use. In addition thereto, the present invention relates to a molding comprising said zeolitic material.

### INTRODUCTION

Zeolitic materials containing another tetravalent element besides Si are known to be efficient catalysts in many applications, including, for example, epoxidation reactions. When conducted in industrial-scale processes, such reactions are typically carried out in continuous mode. Optionally, these zeolitic materials are employed in the form of moldings which, in addition to the catalytically active zeolitic material, comprise a suitable binder.

WO 2011/064191 A1 relates to a process for the preparation of a titanium zeolite catalyst. WO 2021/123227 A1 relates to a continuous synthesis of a titanosilicate zeolitic material. WO 2020/221683 A1 relates to a molding comprising a type MFI zeolitic titanosilicate and a silica binder, its preparation process and use as catalyst. WO 2020/074586 A1 relates to a molding comprising a zeolitic material having framework type MFI.

Xiujuan Deng et al (“Low-Cost Synthesis of Titanium Silicalite-1 (TS-1) with Highly Catalytic Oxidation Performance through a Controlled Hydrolysis Process”, INDUSTRIAL & ENGINEERING CHEMISTRY RESEARCH, vol. 52, no. 3, 23 Jan. 2013 (2013-01-23), pages 1190-1196) discloses a titanium silicate-1 (TS-1) synthesis in low molar ratio of TPAOH/SiO₂ by two-step and multistep hydrolysis process.

Zhang Jian Hui et al (“Synthesis of nanosized TS-1 zeolites through solid transformation method with unprecedented low usage of tetrapropylammonium hydroxide”, MICROPOROUS AND MESOPOROUS MATERIALS, vol. 217, pages 96-101) discloses a synthesis of nanosized TS-1 zeolites in low molar ratio of TPAOH/SiO₂ by solid transformation process.

It was an object of the present invention to provide a novel process for the preparation of a zeolitic material, wherein the framework structure of the zeolitic material comprises Si, O, and a tetravalent element M other than Si, wherein M is selected from the group consisting of Ti, Sn, Zr, Ge, and mixtures of two or more thereof. Further, it was an object to provide a novel zeolitic material, wherein the framework structure of the zeolitic material comprises Si, O, and a tetravalent element M other than Si, wherein M is selected from the group consisting of Ti, Sn, Zr, Ge, and mixtures of two or more thereof, having advantageous characteristics when used as a catalyst or catalyst component, in particular in the epoxidation reaction of propene to propylene oxide. It was a further object to provide a molding comprising said zeolitic material. In addition thereto, it was an object to provide an improved process for the preparation of propylene oxide.

### DETAILED DESCRIPTION

Surprisingly, it was found that an improved process for the preparation of a zeolitic material can be provided wherein one or more sources of SiO₂ are specifically added to a reaction mixture in two or more steps. Furthermore, it was found that an improved zeolitic material can be prepared by said process, wherein said zeolitic material comprises Si, O, and a tetravalent element M other than Si, wherein M is selected from the group consisting of Ti, Sn, Zr, Ge, and mixtures of two or more thereof. In particular, it has surprisingly been found that a zeolitic material as prepared according to the inventive process shows specific properties making it a potent material for use in catalytic reactions. In particular, it has been found that a zeolitic material can be produced displaying unique physical and chemical properties, and that said zeolitic material shows an improved catalytic activity, in particular in the conversion of propylene to propylene oxide.

Therefore, the present invention relates to a process for the production of a zeolitic material, wherein the framework structure of the zeolitic material comprises Si, O, and a tetravalent element M other than Si, wherein M is selected from the group consisting of Ti, Sn, Zr, Ge, and mixtures of two or more thereof, preferably of a zeolitic material according to any of the particular and preferred embodiments of the present invention, the process comprising
(1) preparing a mixture comprising one or more sources of MO₂, one or more organotemplates, a first portion of one or more sources of SiO₂, and a protic solvent system comprising water;
(2) heating the mixture obtained in (1) at a temperature comprised in the range of from 30° C. to the boiling point of the mixture, preferably in the range of from 50 to 95° C., more preferably in the range of from 65 to 90° C., and more preferably in the range of from 75 to 85° C.;
(3) heating the mixture obtained in (2) under autogenous pressure at a temperature comprised in the range of from 100 to 250° C., preferably from 140 to 200° C., more preferably from 155 to 185° C., and more preferably from 165 to 175° C.;
wherein during the course of (2), a second portion of one or more sources of SiO₂ is added to the mixture.

It is preferred that heating in (2) is conducted under atmospheric pressure.

It is preferred that heating in (3) is conducted for a duration in the range of from 0.25 to 10 d, preferably in the range of from 0.5 to 5 d, more preferably in the range of from 1.5 to 2.5 d, more preferably in the range of from 1.75 to 2.25 d.

It is preferred that the one or more sources of SiO₂ comprises one or more alkyoxysilanes, preferably one or more tetraalkoxysilanes, more preferably one or more tetraalkoxysilanes selected from the group consisting of (C₁-C₆) tetraalkoxysilanes and mixtures of two or more thereof, more preferably one or more tetraalkoxysilanes selected from the group consisting of (C₁-C₅) tetraalkoxysilanes and mixtures of two or more thereof, more preferably one or more tetraalkoxysilanes selected from the group consisting of (C₁-C₄) tetraalkoxysilanes and mixtures of two or more thereof, more preferably one or more tetraalkoxysilanes selected from the group consisting of (C₁-C₃) tetraalkoxysilanes and mixtures of two or more thereof, more preferably one or more of tetramethoxysilane and tetraethoxysilane, wherein the one or more sources of Si more preferably comprises tetraethoxysilane, and

wherein the heating of the mixture in (2) includes the hydrolysis of at least a portion of the one or more alkoxysilanes and the distillation of the resulting one or more alcohols from the mixture.

It is preferred that M is selected from the group consisting of Ti, Sn, and a mixture thereof, wherein M preferably comprises Ti, wherein more preferably M is Ti.

It is preferred that in (1) the one or more organotemplates are selected from the group consisting of tetraalkylammonium cation R¹R²R³R⁴N⁺-containing compounds and mixtures thereof, wherein R¹, R², R³ and R⁴ independently from one another stand for alkyl.

In case where in (1) the one or more organotemplates are selected from the group consisting of tetraalkylammonium cation R¹R²R³R⁴N⁺-containing compounds and mixtures thereof, it is preferred that R¹, R², R³, and R⁴ of the one or more tetraalkylammonium cation R¹R²R³R⁴N⁺-containing compounds independently from one another stand for optionally branched (C₁-C₆) alkyl, preferably (C₁-C₅) alkyl, more preferably (C₂-C₄) alkyl, and more preferably for optionally branched (C₂-C₃) alkyl, wherein more preferably R¹, R², R³, and R⁴ independently from one another stand for ethyl or propyl, wherein more preferably R¹, R², R³, and R⁴ of the one or more tetraalkylammonium cation R¹R²R³R⁴N⁺-containing compounds stand for propyl, preferably for n-propyl. Furthermore and independently thereof, it is preferred that independently of one another the one or more tetraalkylammonium cation R¹R²R³R⁴N⁺-containing compounds are salts, preferably one or more salts selected from the group consisting of halides, preferably chloride and/or bromide, more preferably chloride, hydroxide, sulfate, nitrate, phosphate, acetate, and mixtures of two or more thereof, more preferably from the group consisting of chloride, hydroxide, sulfate, and mixtures of two or more thereof, wherein more preferably the one or more tetraalkylammonium cation R¹R²R³R⁴N⁺-containing compounds are tetraalkylammonium hydroxides and/or chlorides, and more preferably tetraalkylammonium hydroxides.

It is preferred that M comprises, preferably is, Ti, and wherein the one or more sources of TiO₂ are selected from the group consisting of oxides and salts of Ti, including mixtures thereof, wherein the one or more sources of TiO₂ preferably comprise one or more compounds selected from the group consisting of titanium oxides, titanium salts, titanyl compounds, titanic acids, titanic acid esters, and mixtures of two or more thereof, more preferably one or more compounds selected from the group consisting of tetrabutyl orthotitanate, tetraisopropyl orthotitanate, tetraethyl orthotitanate, titanium dioxide, titanium tetrachloride, titanium tert-butoxide, TiOSO₄ and/or KTiOPO₄, and mixtures of two or more thereof, more preferably from the group consisting of tetrabutyl orthotitanate, tetraisopropyl orthotitanate, tetraethyl orthotitanate, tetramethyl orthotitanate, titanium dioxide, titanium tetrachloride, titanium tert-butoxide, and mixtures of two or more thereof, wherein the one or more sources of TiO₂ more preferably comprise titanium tert-butoxide, wherein more preferably titanium tert-butoxide is employed as the one or more sources of TiO₂.

It is preferred that the mixture obtained in (2) has a molar ratio of the one or more sources of SiO₂, calculated as SiO₂, to the one or more tetraalkylammonium cation R1R2R3R4N⁺-containing compounds in the range of from 1:0.20 to 1:0.05, preferably in the range of from 1:0.15 to 1:0.09, more preferably in the range of from to 1:0.13 to 1:0.11.

It is preferred that the mixture obtained in (2) has a molar ratio of the one or more sources of SiO₂, calculated as SiO₂, to water in the range of from 1:30 to 1:5, preferably in the range of from 1:20 to 1:12, more preferably in the range of from to 1:17 to 1:15.

It is preferred that the mixture obtained in (2) has a molar ratio of the one or more sources of SiO₂, calculated as SiO₂, to the one or more sources of MO₂, calculated as MO₂, in the range of from 1:0.0100 to 1:0.0010, preferably in the range of from 1:0.0040 to 1:0.0020, more preferably in the range of from to 1:0.0035 to 1:0.0031.

It is preferred that during the course of (2), the second portion of one or more sources of SiO₂ is added continuously or incrementally, preferably incrementally.

In case where during the course of (2), the second portion of one or more sources of SiO₂ is added continuously or incrementally, it is preferred that during (2), the second portion of one or more sources of SiO₂ is incrementally added in 1 to 20 steps, preferably in 1 to 10 steps, more preferably in 1 to 5 steps, more preferably in 1 step.

It is preferred that heating in (2) is conducted continuously or intermittently, preferably intermittently.

In case where heating in (2) is conducted intermittently, it is preferred that the heating consists of two or more heating phases, wherein the second portion of one or more sources of SiO₂ is added during the one or more intervals in between the two or more heating phases, wherein heating in (2) preferably consists of two heating phases, wherein the second portion of one or more sources of SiO₂ is added during the interval in between the two heating phases.

In case where heating in (2) is conducted intermittently, it is particularly preferred that the heating in (2) consists of three or more heating phases, wherein the second portion of one or more sources of SiO₂ is added in equal sub-portions during each of the respective intervals in between the three or more heating phases, wherein each sub-portion corresponds to a fraction 1/n of the total amount of the second portion, wherein n stands for the number of intervals during which the respective sub-portions are respectively added, and n+1 corresponds to the number of heating steps, wherein n is an integer comprised in the range of from 2 to 5, wherein n is preferably 2 or 3, more preferably 2.

In case where heating in (2) is conducted intermittently, it is preferred that during the respective interval, during which the second portion or a sub-portion of the second portion of one or more sources of SiO₂ is added, the mixture has a temperature comprised in the range of from 15 to 65° C., preferably in the range of from 20 to 60° C., more preferably in the range of from 35 to 55° C., more preferably in the range of from 48 to 52° C. Furthermore and independently thereof, it is preferred that the respective heating phases independently from one another are conducted for a duration comprised in the range of from 0.1 to 5 h, preferably in the range of from 0.5 to 3.5 h, more preferably in the range of from 1.75 to 2.25 h.

In case where heating in (2) is conducted intermittently, it is preferred that heating consists of 2 to 6 heating phases, preferably of 2 to 4 heating phases, more preferably of 2 or 3 heating phases, wherein heating more preferably consists of 2 heating phases.

It is preferred that the first portion of one or more sources of SiO₂ comprises an amount in the range of from 45 to 90 mol-%, calculated as SiO₂, preferably in the range of from 60 to 75 mol-%, more preferably in the range of from 65 to 68 mol.-% of the total amount (100 mol.-%) of the one or more sources of SiO₂, calculated as SiO₂, added to the mixture in (1) and during the course of (2).

It is preferred that the second portion of one or more sources of SiO₂ comprises an amount in the range of from 10 to 55 mol-%, calculated as SiO₂, preferably in the range of from 25 to 40 mol-%, more preferably in the range of from 32 to 35 mol.-% of the total amount (100 mol.-%) of the one or more sources of SiO₂, calculated as SiO₂, added to the mixture in (1) and during the course of (2).

It is preferred that the mixture obtained in (2) is heated in (3) under a pressure in the range of from 0.5 to 15 MPa, preferably in the range of from 1 to 10 MPa, more preferably in the range of from 2 to 6 MPa, more preferably in the range of from 3 to 5 MPa.

It is preferred that the process further comprises one or more of
(4) isolating the zeolitic material obtained in (3);
(5) optionally washing the zeolitic material obtained in (3) or (4);
(6) drying the zeolitic material obtained in (3), (4) or (5);
(7) calcining the zeolitic material obtained in (3), (4), (5) or (6).

It is preferred that in (4) isolating the zeolitic material obtained in (3) is effected by one or more of centrifugation and filtration.

It is preferred that in (5) the zeolitic material obtained in (3) or (4) is washed with a polar protic solvent system, preferably with water, more preferably with de-ionized water.

It is preferred that drying in (6) is effected at a temperature in the range of from 50 to 150° C., more preferably from 70 to 130° C., more preferably from 80 to 120° C., and more preferably from 90 to 110° C.

It is preferred that calcining in (7) is effected at a temperature in the range of from 300 to 700° C., preferably from 400 to 625° C., more preferably from 500 to 600° C., more preferably from 525 to 575° C., and more preferably from 540 to 560° C.

It is preferred that the zeolitic material has a framework structure type selected from the group consisting of MFI, MEL, IMF, SVY, FER, SVR, and an intergrowth structure of two or more thereof, preferably selected from the group consisting of MFI, MEL, and an intergrowth structure thereof, wherein the zeolitic material more preferably has an MFI framework structure type.

The present invention also relates to a zeolitic material obtainable and/or obtained by the process according to any one of the particular and preferred embodiments of the present invention.

The present invention also relates to a zeolitic material, preferably obtained or obtainable according to any one of the particular and preferred embodiments the inventive process, wherein the framework structure of the zeolitic material comprises Si, O, and a tetravalent element M other than Si, wherein M is selected from the group consisting of Ti, Sn, Zr, Ge, and mixtures of two or more thereof, and wherein the UV-vis spectrum of the zeolitic material displays a first absorption band A1 having a maximum in the range of from 180 to 230 nm, preferably in the range of from 200 to 210 nm and a second absorption band A2 having a maximum in the range of from 290 to 370 nm, preferably in the range of from 310 to 350 nm, wherein the UV-vis spectrum is preferably determined according to Reference Example 1.8.

It is preferred that the UV-vis spectrum displays no further maximum between the maximum of the first absorption band and the maximum of the second absorption band.

It is preferred that the FTIR spectrum of the zeolitic material displays a first absorption band B1 having a maximum in the range of from 3,450 to 3,550 cm-1, preferably in the range of from 3,475 to 3,525 cm⁻¹,

and a second absorption band B2 having a maximum in the range of from 3,700 to 3,775 cm⁻¹, preferably in the range of from 3,725 to 3,740 cm⁻¹,
wherein the intensity ratio B1:B2 of the first absorption band to the second absorption band is comprised in the range of from 0.70:1 to 0.90:1, preferably in the range of from 0.73:1 to 0.88:1, wherein the FTIR spectrum is preferably determined according to Reference Example 1.7.

Furthermore, it is preferred that the zeolitic material of the present invention has a molar ratio of Si to M in the range of from 10 to 75, preferably in the range of from 30 to 60, more preferably in the range of from 35 to 55, more preferably in the range of from 40 to 50, more preferably in the range of from 43 to 45.

It is also preferred that the zeolitic material of the present invention has a BET specific surface area in the range of from 350 to 510 m²/g, preferably in the range of from 400 to 460 m²/g, preferably in the range of from 411 to 448 m²/g, more preferably in the range of from 419 to 440 m²/g, wherein the BET specific surface area is preferably determined according to ISO 9277:2010.

It is also preferred that the zeolitic material of the present invention has micropore volume in the range of from 0.160 to 0.210 cm³/g, preferably in the range of from 0.173 to 0.193 cm³/g, more preferably in the range of from 0.179 to 0.187 cm³/g, more preferably in the range of from 0.181 to 0.185 cm³/g, wherein the micropore volume is preferably determined according to ISO 15901-1:2016.

It is also preferred that the zeolitic material of the present invention shows in the temperature programmed desorption of ammonia (NH₃-TPD) profile a total amount of acid sites in the range of from 0.020 to 0.070 mmol/g, preferably in the range of from 0.032 to 0.056 mmol/g, more preferably in the range of from 0.038 to 0.050 mmol/g, more preferably in the range of from 0.042 to 0.046 mmol/g, wherein the temperature programmed desorption of ammonia (NH₃-TPD) profile is preferably determined according to Reference Example 1.5.

It is also preferred that the zeolitic material of the present invention shows in the temperature programmed desorption of ammonia (NH₃-TPD) profile a band having a maximum in the range of from 130 to 190° C., preferably in the range of from 140 to 180° C.

Furthermore, it is preferred that the zeolitic material of the present invention has a water adsorption in the range of from 5 to 20 weight-%, preferably in the range of from 10 to 19 weight-%, more preferably in the range of from 12 to 17 weight-%, more preferably in the range of from 14.0 to 15.0 weight-%, more preferably in the range of from 14.1 to 14.5 weight-%, when exposed to a relative humidity of 91%, wherein the water adsorption is preferably determined according to Reference Example 1.4.

Furthermore, it is preferred that the zeolitic material of the present invention has a framework structure type selected from the group consisting of MFI, MEL, IMF, SVY, FER, SVR, and an intergrowth structure of two or more thereof, preferably selected from the group consisting of MFI, MEL, and an intergrowth structure thereof, wherein the zeolitic material more preferably has a MFI framework structure type.

It is preferred that M is selected from the group consisting of Ti, Sn, and mixtures of two or more thereof, wherein M is preferably Ti or Sn, wherein M more preferably is Ti.

It is preferred that M is Ti, and wherein the zeolitic material has a framework structure type MFI, wherein the zeolitic material is more preferably a TS-1 zeolite.

The present invention also relates to a process for preparing a molding, comprising
(A) providing a zeolitic material according to any one of the particular and preferred embodiments of the present invention;
(B) mixing the zeolitic material provided in step (A) with one or more binders;
(C) optionally kneading of the mixture obtained in step (B);
(D) molding of the mixture obtained in step (B) or (C) to obtain one or more moldings;
(E) drying of the one or more moldings obtained in step (D); and
(F) calcining of the dried molding obtained in step (E).

It is preferred that the one or more binders in (B) are selected from the group consisting of inorganic binders, wherein the one or more binders preferably comprise one or more sources of a metal oxide and/or of a metalloid oxide, more preferably one or more sources of a metal oxide and/or of a metalloid oxide selected from the group consisting of silica, alumina, titania, zirconia, lanthana, magnesia, and mixtures and/or mixed oxides of two or more thereof, more preferably from the group consisting of silica, alumina, titania, zirconia, magnesia, silica-alumina mixed oxides, silica-titania mixed oxides, silica-zirconia mixed oxides, silica-lanthana mixed oxides, silica-zirconia-lanthana mixed oxides, alumina-titania mixed oxides, alumina-zirconia mixed oxides, alumina-lanthana mixed oxides, alumina-zirconia-lanthana mixed oxides, titania-zirconia mixed oxides, and mixtures and/or mixed oxides of two or more thereof, more preferably from the group consisting of silica, alumina, silica-alumina mixed oxides, and mixtures of two or more thereof, wherein more preferably the one or more binders in (B) comprise one or more sources of silica, wherein more preferably the one or more binders in (B) consist of one or more sources of silica, wherein the one or more sources of silica preferably comprise one or more compounds selected from the group consisting of fumed silica, colloidal silica, silica-alumina, colloidal silica-alumina, and mixtures of two or more thereof, more preferably one or more compounds selected from the group consisting of fumed silica, colloidal silica, and mixtures thereof, wherein more preferably the one or more binders in (B) consist of colloidal silica.

It is preferred that step (B) further comprises mixing the zeolitic material and the one or more binders with a solvent system, wherein the solvent system comprises one or more solvents, wherein preferably the solvent system comprises one or more hydrophilic solvents, the hydrophilic solvents preferably being selected from the group consisting of polar solvents, more preferably from the group consisting of polar protic solvents, wherein more preferably the solvent system comprises one or more polar protic solvents selected from the group consisting of water, alcohols, carboxylic acids, and mixtures of two or more thereof, more preferably from the group consisting of water, C1-C5 alcohols, C1-C5 carboxylic acids, and mixtures of two or more thereof, more preferably from the group consisting of water, C1-C4 alcohols, C1-C4 carboxylic acids, and mixtures of two or more thereof, more preferably from the group consisting of water, C1-C3 alcohols, C1-C3 carboxylic acids, and mixtures of two or more thereof, more preferably from the group consisting of water, methanol, ethanol, propanol, formic acid, acetic acid, and mixtures of two or more thereof, more preferably from the group consisting of water, ethanol, acetic acid, and mixtures of two or more thereof, wherein more preferably the solvent system comprises water and/or ethanol, and wherein more preferably the solvent system comprises water, wherein even more preferably the solvent system consists of water.

It is preferred that step (B) further comprises mixing the zeolitic material and the one or more binders with one or more pore forming agents and/or lubricants and/or plasticizers, wherein the one or more pore forming agents and/or lubricants and/or plasticizers are preferably selected from the group consisting of polymers, carbohydrates, graphite, plant additives, and mixtures of two or more thereof, more preferably from the group consisting of polymeric vinyl compounds, polyalkylene oxides, polyacrylates, polyolefins, polyamides, polyesters, cellulose and cellulose derivatives, sugars, Sesbania cannabina, and mixtures of two or more thereof, more preferably from the group consisting of polystyrene, C2-C3 polyalkylene oxides, cellulose derivatives, sugars, and mixtures of two or more thereof, more preferably from the group consisting of polystyrene, polyethylene oxide, C1-C2 hydroxyalkylated and/or C1-C2 alkylated cellulose derivatives, sugars, and mixtures of two or more thereof, more preferably from the group consisting of polystyrene, polyethylene oxide, hydroxyethyl methyl cellulose, and mixtures of two or more thereof, wherein more preferably the one or more pore forming agents and/or lubricants and/or plasticizers consists of one or more selected from the group consisting of polystyrene, polyethylene oxide, hydroxyethyl methyl cellulose, and mixtures of two or more thereof, and more preferably wherein the one or more pore forming agents and/or lubricants and/or plasticizers consist of a mixture of polystyrene, polyethylene oxide, and hydroxyethyl methyl cellulose.

It is preferred that the calcining of the dried molding obtained in step (E) is performed at a temperature ranging from 350 to 850° C., preferably from 400 to 700° C., more preferably from 450 to 650° C., and more preferably from 475 to 600° C.

The present invention also relates to a molding obtainable or obtained by the process of any one of the particular and preferred embodiments of the present invention.

The present invention also relates to the use of a zeolitic material according to any one of the particular and preferred embodiments of the present invention, or of a molding according to any one of the particular and preferred embodiments of the present invention, as a catalyst, catalyst component, absorbent, adsorbent, or for ion exchange, preferably as a catalyst and/or catalyst component, more preferably as a catalyst and/or catalyst component in a reaction involving C—C bond formation and/or conversion, and preferably as a catalyst and/or catalyst component in an isomerization reaction, in an ammoxidation reaction, in an amination reaction, in a hydrocracking reaction, in an alkylation reaction, in an acylation reaction, in a reaction for the conversion of alkanes to olefins, or in a reaction for the conversion of one or more oxygenates to olefins and/or aromatics, in a reaction for the synthesis of hydrogen peroxide, in an aldol condensation reaction, in a reaction for the isomerization of epoxides, in a transesterification reaction, or in an epoxidation reaction, preferably as a catalyst and/or catalyst component in a reaction for the epoxidation of olefins, more preferably in a reaction for the epoxidation of C2-C5 alkenes, more preferably in a reaction for the epoxidation of C2-C4 alkenes, in a reaction for the epoxidation of C2 or C3 alkenes, more preferably for the epoxidation of C3 alkenes, and more preferably as a catalyst or catalyst component for the conversion of propylene to propylene oxide.

The present invention is further illustrated by the following set of embodiments and combinations of embodiments resulting from the dependencies and back-references as indicated. In particular, it is noted that in each instance where a range of embodiments is mentioned, for example in the context of a term such as “The process of any one of embodiments 1 to 4”, every embodiment in this range is meant to be explicitly disclosed for the skilled person, i.e. the wording of this term is to be understood by the skilled person as being synonymous to “The process of any one of embodiments 1, 2, 3, and 4”. Further, it is explicitly noted that the following set of embodiments is not the set of claims determining the extent of protection, but represents a suitably structured part of the description directed to general and preferred aspects of the present invention.
1. A process for the production of a zeolitic material, wherein the framework structure of the zeolitic material comprises Si, O, and a tetravalent element M other than Si, wherein M is selected from the group consisting of Ti, Sn, Zr, Ge, and mixtures of two or more thereof, preferably of a zeolitic material according to any of embodiments 32 to 44, the process comprising
   (1) preparing a mixture comprising one or more sources of MO₂, one or more organotemplates, a first portion of one or more sources of SiO₂, and a protic solvent system comprising water;
   (2) heating the mixture obtained in (1) at a temperature comprised in the range of from 30° C. to the boiling point of the mixture, preferably in the range of from 50 to 95° C., more preferably in the range of from 65 to 90° C., and more preferably in the range of from 75 to 85° C.;
   (3) heating the mixture obtained in (2) under autogenous pressure at a temperature comprised in the range of from 100 to 250° C., preferably from 140 to 200° C., more preferably from 155 to 185° C., and more preferably from 165 to 175° C.;
   wherein during the course of (2), a second portion of one or more sources of SiO₂ is added to the mixture.
2. The process of embodiment 1, wherein heating in (2) is conducted under atmospheric pressure.
3. The process of embodiment 1 or 2, wherein heating in (3) is conducted for a duration in the range of from 0.25 to 10 d, preferably in the range of from 0.5 to 5 d, more preferably in the range of from 1.5 to 2.5 d, more preferably in the range of from 1.75 to 2.25 d.
4. The process of any of embodiments 1 to 3, wherein the one or more sources of SiO₂ comprises one or more alkyoxysilanes, preferably one or more tetraalkoxysilanes, more preferably one or more tetraalkoxysilanes selected from the group consisting of (C₁-C₆) tetraalkoxysilanes and mixtures of two or more thereof, more preferably one or more tetraalkoxysilanes selected from the group consisting of (C₁-C₅) tetraalkoxysilanes and mixtures of two or more thereof, more preferably one or more tetraalkoxysilanes selected from the group consisting of (C₁-C₄) tetraalkoxysilanes and mixtures of two or more thereof, more preferably one or more tetraalkoxysilanes selected from the group consisting of (C₁-C₃) tetraalkoxysilanes and mixtures of two or more thereof, more preferably one or more of tetramethoxysilane and tetraethoxysilane, wherein the one or more sources of Si more preferably comprises tetraethoxysilane, and
   wherein the heating of the mixture in (2) includes the hydrolysis of at least a portion of the one or more alkoxysilanes and the distillation of the resulting one or more alcohols from the mixture.
5. The process of any of embodiments 1 to 4, wherein M is selected from the group consisting of Ti, Sn, and a mixture thereof, wherein M preferably comprises Ti, wherein more preferably M is Ti.
6. The process of any of embodiments 1 to 5, wherein in (1) the one or more organotemplates are selected from the group consisting of tetraalkylammonium cation R¹R²R³R⁴N⁺-containing compounds and mixtures thereof, wherein R¹, R², R³ and R⁴ independently from one another stand for alkyl.
7. The process of any embodiment 6, wherein R¹, R², R³, and R⁴ of the one or more tetraalkylammonium cation R¹R²R³R⁴N⁺-containing compounds independently from one another stand for optionally branched (C₁-C₆) alkyl, preferably (C₁-C₅) alkyl, more preferably (C₂-C₄) alkyl, and more preferably for optionally branched (C₂-C₃) alkyl, wherein more preferably R¹, R², R³, and R⁴ independently from one another stand for ethyl or propyl, wherein more preferably R¹, R², R³, and R⁴ of the one or more tetraalkylammonium cation R¹R²R³R⁴N⁺-containing compounds stand for propyl, preferably for n-propyl.
8. The process of embodiment 5 or 6, wherein independently of one another the one or more tetraalkylammonium cation R¹R²R³R⁴N⁺-containing compounds are salts, preferably one or more salts selected from the group consisting of halides, preferably chloride and/or bromide, more preferably chloride, hydroxide, sulfate, nitrate, phosphate, acetate, and mixtures of two or more thereof, more preferably from the group consisting of chloride, hydroxide, sulfate, and mixtures of two or more thereof, wherein more preferably the one or more tetraalkylammonium cation R¹R²R³R⁴N⁺-containing compounds are tetraalkylammonium hydroxides and/or chlorides, and more preferably tetraalkylammonium hydroxides.
9. The process of any one of embodiments 1 to 8, wherein M comprises, preferably is, Ti, and wherein the one or more sources of TiO₂ are selected from the group consisting of oxides and salts of Ti, including mixtures thereof,
   wherein the one or more sources of TiO₂ preferably comprise one or more compounds selected from the group consisting of titanium oxides, titanium salts, titanyl compounds, titanic acids, titanic acid esters, and mixtures of two or more thereof, more preferably one or more compounds selected from the group consisting of tetrabutyl orthotitanate, tetraisopropyl orthotitanate, tetraethyl orthotitanate, titanium dioxide, titanium tetrachloride, titanium tert-butoxide, TiOSO₄ and/or KTiOPO₄, and mixtures of two or more thereof, more preferably from the group consisting of tetrabutyl orthotitanate, tetraisopropyl orthotitanate, tetraethyl orthotitanate, tetramethyl orthotitanate, titanium dioxide, titanium tetrachloride, titanium tert-butoxide, and mixtures of two or more thereof, wherein the one or more sources of TiO₂ more preferably comprise titanium tert-butoxide, wherein more preferably titanium tert-butoxide is employed as the one or more sources of TiO₂.
10. The process of any one of embodiments 1 to 9, wherein the mixture obtained in (2) has a molar ratio of the one or more sources of SiO₂, calculated as SiO₂, to the one or more tetraalkylammonium cation R¹R²R³R⁴N⁺-containing compounds in the range of from 1:0.20 to 1:0.05, preferably in the range of from 1:0.15 to 1:0.09, more preferably in the range of from to 1:0.13 to 1:0.11.
11. The process of any one of embodiments 1 to 10, wherein the mixture obtained in (2) has a molar ratio of the one or more sources of SiO₂, calculated as SiO₂, to water in the range of from 1:30 to 1:5, preferably in the range of from 1:20 to 1:12, more preferably in the range of from to 1:17 to 1:15.
12. The process of any one of embodiments 1 to 11, wherein the mixture obtained in (2) has a molar ratio of the one or more sources of SiO₂, calculated as SiO₂, to the one or more sources of MO₂, calculated as MO₂, in the range of from 1:0.0100 to 1:0.0010, preferably in the range of from 1:0.0040 to 1:0.0020, more preferably in the range of from to 1:0.0035 to 1:0.0031.
13. The process of any one of embodiments 1 to 12, wherein during the course of (2), the second portion of one or more sources of SiO₂ is added continuously or incrementally, preferably incrementally.
14. The process of embodiment 13, wherein during (2), the second portion of one or more sources of SiO₂ is incrementally added in 1 to 20 steps, preferably in 1 to 10 steps, more preferably in 1 to 5 steps, more preferably in 1 step.
15. The process of any one of embodiments 1 to 14, wherein heating in (2) is conducted continuously or intermittently, preferably intermittently.
16. The process of embodiment 15, wherein heating in (2) is conducted intermittently, wherein the heating consists of two or more heating phases, wherein the second portion of one or more sources of SiO₂ is added during the one or more intervals in between the two or more heating phases, wherein heating in (2) preferably consists of two heating phases, wherein the second portion of one or more sources of SiO₂ is added during the interval in between the two heating phases.
17. The process of embodiment 16, wherein the heating in (2) consists of three or more heating phases, wherein the second portion of one or more sources of SiO₂ is added in equal sub-portions during each of the respective intervals in between the three or more heating phases, wherein each sub-portion corresponds to a fraction 1/n of the total amount of the second portion, wherein n stands for the number of intervals during which the respective sub-portions are respectively added, and n+1 corresponds to the number of heating steps, wherein n is an integer comprised in the range of from 2 to 5, wherein n is preferably 2 or 3, more preferably 2.
18. The process of embodiment 16 or 17, wherein during the respective interval, during which the second portion or a sub-portion of the second portion of one or more sources of SiO₂ is added, the mixture has a temperature comprised in the range of from 15 to 65° C., preferably in the range of from 20 to 60° C., more preferably in the range of from 35 to 55° C., more preferably in the range of from 48 to 52° C.
19. The process of any one of embodiments 16 to 18, wherein the respective heating phases independently from one another are conducted for a duration comprised in the range of from 0.1 to 5 h, preferably in the range of from 0.5 to 3.5 h, more preferably in the range of from 1.75 to 2.25 h.
20. The process of any one of embodiments 16 to 19, wherein heating consists of 2 to 6 heating phases, preferably of 2 to 4 heating phases, more preferably of 2 or 3 heating phases, wherein heating more preferably consists of 2 heating phases.
21. The process of any one of embodiments 1 to 20, wherein the first portion of one or more sources of SiO₂ comprises an amount in the range of from 45 to 90 mol-%, calculated as SiO₂, preferably in the range of from 60 to 75 mol-%, more preferably in the range of from 65 to 68 mol.-% of the total amount (100 mol.-%) of the one or more sources of SiO₂, calculated as SiO₂, added to the mixture in (1) and during the course of (2).
22. The process of any one of embodiments 1 to 21, wherein the second portion of one or more sources of SiO₂ comprises an amount in the range of from 10 to 55 mol-%, calculated as SiO₂, preferably in the range of from 25 to 40 mol-%, more preferably in the range of from 32 to 35 mol.-% of the total amount (100 mol.-%) of the one or more sources of SiO₂, calculated as SiO₂, added to the mixture in (1) and during the course of (2).
23. The process of any one of embodiments 1 to 22, wherein the mixture obtained in (2) is heated in (3) under a pressure in the range of from 0.5 to 15 MPa, preferably in the range of from 1 to 10 MPa, more preferably in the range of from 2 to 6 MPa, more preferably in the range of from 3 to 5 MPa.
24. The process of any one of embodiments 1 to 23, further comprising one or more of
   (4) isolating the zeolitic material obtained in (3);
   (5) optionally washing the zeolitic material obtained in (3) or (4);
   (6) drying the zeolitic material obtained in (3), (4) or (5);
   (7) calcining the zeolitic material obtained in (3), (4), (5) or (6).
25. The process of embodiment 24, wherein in (4) isolating the zeolitic material obtained in (3) is effected by one or more of centrifugation and filtration.
26. The process of embodiment 24 or 25, wherein in (5) the zeolitic material obtained in (3) or (4) is washed with a polar protic solvent system, preferably with water, more preferably with de-ionized water.
27. The process of any one of embodiments 24 to 26, wherein drying in (6) is effected at a temperature in the range of from 50 to 150° C., more preferably from 70 to 130° C., more preferably from 80 to 120° C., and more preferably from 90 to 110° C.
28. The process of any one of embodiments 24 to 27, wherein calcining in (7) is effected at a temperature in the range of from 300 to 700° C., preferably from 400 to 625° C., more preferably from 500 to 600° C., more preferably from 525 to 575° C., and more preferably from 540 to 560° C.
29. The process of any one of embodiments 1 to 28, wherein the zeolitic material has a framework structure type selected from the group consisting of MFI, MEL, IMF, SVY, FER, SVR, and an intergrowth structure of two or more thereof, preferably selected from the group consisting of MFI, MEL, and an intergrowth structure thereof, wherein the zeolitic material more preferably has an MFI framework structure type.
30. A zeolitic material obtainable and/or obtained by the process according to any one of embodiments 1 to 29.
31. A zeolitic material, preferably obtained or obtainable according to the process of any one of embodiments 1 to 29, wherein the framework structure of the zeolitic material comprises Si, O, and a tetravalent element M other than Si, wherein M is selected from the group consisting of Ti, Sn, Zr, Ge, and mixtures of two or more thereof, and wherein the UV-vis spectrum of the zeolitic material displays a first absorption band A1 having a maximum in the range of from 180 to 230 nm, preferably in the range of from 200 to 210 nm and a second absorption band A2 having a maximum in the range of from 290 to 370 nm, preferably in the range of from 310 to 350 nm, wherein the UV-vis spectrum is preferably determined according to Reference Example 1.8.
32. The zeolitic material of embodiment 31, wherein the UV-vis spectrum displays no further maximum between the maximum of the first absorption band and the maximum of the second absorption band.
33. The zeolitic material of embodiment 31 or 32, wherein the FTIR spectrum of the zeolitic material displays a first absorption band B1 having a maximum in the range of from 3,450 to 3,550 cm-1, preferably in the range of from 3,475 to 3,525 cm-1, and a second absorption band B2 having a maximum in the range of from 3,700 to 3,775 cm-1, preferably in the range of from 3,725 to 3,740 cm-1, wherein the intensity ratio B1:B2 of the first absorption band to the second absorption band is comprised in the range of from 0.70:1 to 0.90:1, preferably in the range of from 0.73:1 to 0.88:1, wherein the FTIR spectrum is preferably determined according to Reference Example 1.7.
34. The zeolitic material of any one of embodiments 31 to 33, having a molar ratio of Si to M in the range of from 10 to 75, preferably in the range of from 30 to 60, more preferably in the range of from 35 to 55, more preferably in the range of from 40 to 50, more preferably in the range of from 43 to 45.
35. The zeolitic material of any one of embodiments 31 to 34, having a BET specific surface area in the range of from 350 to 510 m²/g, preferably in the range of from 400 to 460 m²/g, preferably in the range of from 411 to 448 m²/g, more preferably in the range of from 419 to 440 m²/g, wherein the BET specific surface area is preferably determined according to ISO 9277:2010.
36. The zeolitic material of any one of embodiments 31 to 35, having micropore volume in the range of from 0.160 to 0.210 cm³/g, preferably in the range of from 0.173 to 0.193 cm³/g, more preferably in the range of from 0.179 to 0.187 cm³/g, more preferably in the range of from 0.181 to 0.185 cm³/g, wherein the micropore volume is preferably determined according to ISO 15901-1:2016.
37. The zeolitic material of any one of embodiments 31 to 36, showing in the temperature programmed desorption of ammonia (NH₃-TPD) profile a total amount of acid sites in the range of from 0.020 to 0.070 mmol/g, preferably in the range of from 0.032 to 0.056 mmol/g, more preferably in the range of from 0.038 to 0.050 mmol/g, more preferably in the range of from 0.042 to 0.046 mmol/g, wherein the temperature programmed desorption of ammonia (NH₃-TPD) profile is preferably determined according to Reference Example 1.5.
38. The zeolitic material of any one of embodiments 31 to 37, showing in the temperature programmed desorption of ammonia (NH₃-TPD) profile a band having a maximum in the range of from 130 to 190° C., preferably in the range of from 140 to 180° C.
39. The zeolitic material of any one of embodiments 31 to 38, having a water adsorption in the range of from 5 to 20 weight-%, preferably in the range of from 10 to 19 weight-%, more preferably in the range of from 12 to 17 weight-%, more preferably in the range of from 14.0 to 15.0 weight-%, more preferably in the range of from 14.1 to 14.5 weight-%, when exposed to a relative humidity of 91%, wherein the water adsorption is preferably determined according to Reference Example 1.4.
40. The zeolitic material of any one of embodiments 31 to 39, having a framework structure type selected from the group consisting of MFI, MEL, IMF, SVY, FER, SVR, and an intergrowth structure of two or more thereof, preferably selected from the group consisting of MFI, MEL, and an intergrowth structure thereof, wherein the zeolitic material more preferably has a MFI framework structure type.
41. The zeolitic material of any one of embodiments 31 to 40, wherein M is selected from the group consisting of Ti, Sn, and mixtures of two or more thereof, wherein M is preferably Ti or Sn, wherein M more preferably is Ti.
42. The zeolitic material of any one of embodiments 31 to 41, wherein M is Ti, and wherein the zeolitic material has a framework structure type MFI, wherein the zeolitic material is more preferably a TS-1 zeolite.
43. A process for preparing a molding, comprising
   (A) providing a zeolitic material according to any one of embodiments 30 to 42;
   (B) mixing the zeolitic material provided in step (A) with one or more binders;
   (C) optionally kneading of the mixture obtained in step (B);
   (D) molding of the mixture obtained in step (B) or (C) to obtain one or more moldings;
   (E) drying of the one or more moldings obtained in step (D); and
   (F) calcining of the dried molding obtained in step (E).
44. The process of embodiment 43, wherein the one or more binders in (B) are selected from the group consisting of inorganic binders, wherein the one or more binders preferably comprise one or more sources of a metal oxide and/or of a metalloid oxide, more preferably one or more sources of a metal oxide and/or of a metalloid oxide selected from the group consisting of silica, alumina, titania, zirconia, lanthana, magnesia, and mixtures and/or mixed oxides of two or more thereof, more preferably from the group consisting of silica, alumina, titania, zirconia, magnesia, silica-alumina mixed oxides, silica-titania mixed oxides, silica-zirconia mixed oxides, silica-lanthana mixed oxides, silica-zirconia-lanthana mixed oxides, alumina-titania mixed oxides, alumina-zirconia mixed oxides, alumina-lanthana mixed oxides, alumina-zirconia-lanthana mixed oxides, titania-zirconia mixed oxides, and mixtures and/or mixed oxides of two or more thereof, more preferably from the group consisting of silica, alumina, silica-alumina mixed oxides, and mixtures of two or more thereof, wherein more preferably the one or more binders in (B) comprise one or more sources of silica, wherein more preferably the one or more binders in (B) consist of one or more sources of silica, wherein the one or more sources of silica preferably comprise one or more compounds selected from the group consisting of fumed silica, colloidal silica, silica-alumina, colloidal silica-alumina, and mixtures of two or more thereof, more preferably one or more compounds selected from the group consisting of fumed silica, colloidal silica, and mixtures thereof, wherein more preferably the one or more binders in (B) consist of colloidal silica.
45. The process of embodiment 43 or 44, wherein step (B) further comprises mixing the zeolitic material and the one or more binders with a solvent system, wherein the solvent system comprises one or more solvents, wherein preferably the solvent system comprises one or more hydrophilic solvents, the hydrophilic solvents preferably being selected from the group consisting of polar solvents, more preferably from the group consisting of polar protic solvents, wherein more preferably the solvent system comprises one or more polar protic solvents selected from the group consisting of water, alcohols, carboxylic acids, and mixtures of two or more thereof, more preferably from the group consisting of water, C1-C5 alcohols, C1-C5 carboxylic acids, and mixtures of two or more thereof, more preferably from the group consisting of water, C1-C4 alcohols, C1-C4 carboxylic acids, and mixtures of two or more thereof, more preferably from the group consisting of water, C1-C3 alcohols, C1-C3 carboxylic acids, and mixtures of two or more thereof, more preferably from the group consisting of water, methanol, ethanol, propanol, formic acid, acetic acid, and mixtures of two or more thereof, more preferably from the group consisting of water, ethanol, acetic acid, and mixtures of two or more thereof, wherein more preferably the solvent system comprises water and/or ethanol, and wherein more preferably the solvent system comprises water, wherein even more preferably the solvent system consists of water.
46. The process of any one of embodiments 43 to 45, wherein step (B) further comprises mixing the zeolitic material and the one or more binders with one or more pore forming agents and/or lubricants and/or plasticizers, wherein the one or more pore forming agents and/or lubricants and/or plasticizers are preferably selected from the group consisting of polymers, carbohydrates, graphite, plant additives, and mixtures of two or more thereof, more preferably from the group consisting of polymeric vinyl compounds, polyalkylene oxides, polyacrylates, polyolefins, polyamides, polyesters, cellulose and cellulose derivatives, sugars, Sesbania cannabina, and mixtures of two or more thereof, more preferably from the group consisting of polystyrene, C2-C3 polyalkylene oxides, cellulose derivatives, sugars, and mixtures of two or more thereof, more preferably from the group consisting of polystyrene, polyethylene oxide, C1-C2 hydroxyalkylated and/or C1-C2 alkylated cellulose derivatives, sugars, and mixtures of two or more thereof, more preferably from the group consisting of polystyrene, polyethylene oxide, hydroxyethyl methyl cellulose, and mixtures of two or more thereof, wherein more preferably the one or more pore forming agents and/or lubricants and/or plasticizers consists of one or more selected from the group consisting of polystyrene, polyethylene oxide, hydroxyethyl methyl cellulose, and mixtures of two or more thereof, and more preferably wherein the one or more pore forming agents and/or lubricants and/or plasticizers consist of a mixture of polystyrene, polyethylene oxide, and hydroxyethyl methyl cellulose.
47. The process of any one of embodiments 43 to 46, wherein the calcining of the dried molding obtained in step (E) is performed at a temperature ranging from 350 to 850° C., preferably from 400 to 700° C., more preferably from 450 to 650° C., and more preferably from 475 to 600° C.
48. A molding obtainable or obtained by the process of any one of embodiments 43 to 47.
49. Use of a zeolitic material according to any one of embodiments 30 to 42 or of a molding according to embodiment 48 as a catalyst, catalyst component, absorbent, adsorbent, or for ion exchange, preferably as a catalyst and/or catalyst component, more preferably as a catalyst and/or catalyst component in a reaction involving C—C bond formation and/or conversion, and preferably as a catalyst and/or catalyst component in an isomerization reaction, in an ammoxidation reaction, in an amination reaction, in a hydrocracking reaction, in an alkylation reaction, in an acylation reaction, in a reaction for the conversion of alkanes to olefins, or in a reaction for the conversion of one or more oxygenates to olefins and/or aromatics, in a reaction for the synthesis of hydrogen peroxide, in an aldol condensation reaction, in a reaction for the isomerization of epoxides, in a transesterification reaction, or in an epoxidation reaction, preferably as a catalyst and/or catalyst component in a reaction for the epoxidation of olefins, more preferably in a reaction for the epoxidation of C2-C5 alkenes, more preferably in a reaction for the epoxidation of C2-C4 alkenes, in a reaction for the epoxidation of C2 or C3 alkenes, more preferably for the epoxidation of C3 alkenes, and more preferably as a catalyst or catalyst component for the conversion of propylene to propylene oxide.

The present invention is further illustrated by the following examples, comparative examples and reference examples.

### DESCRIPTION OF THE FIGURES

FIG. 1: shows the XRD of the zeolitic materials of Example 1 and Comparative Example 1. The 2theta angle in degree is shown on the abscissa and the intensity is shown on the ordinate in arbitrary units.

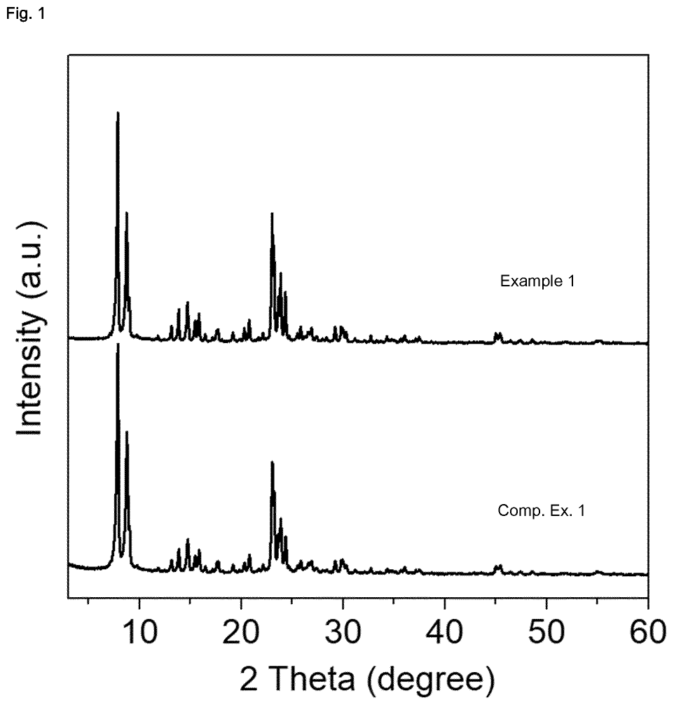

FIGS. 2A and 2B: show the SEM images of the zeolitic materials of Comparative Example 1 and Example 1, respectively.

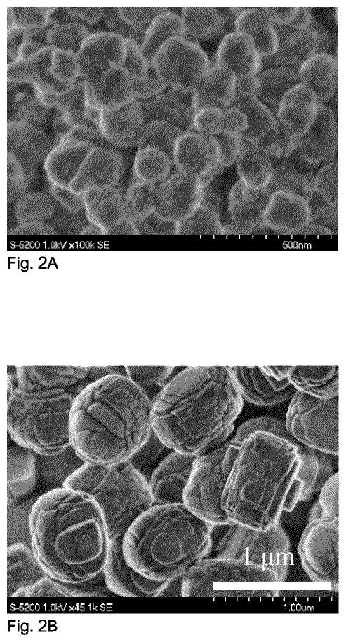

FIG. 3: shows the UV-vis spectra of the zeolitic materials of Example 1 and Comparative Example 1. The wavelength in nm is shown on the abscissa and the absorbance is shown in arbitrary units on the ordinate.

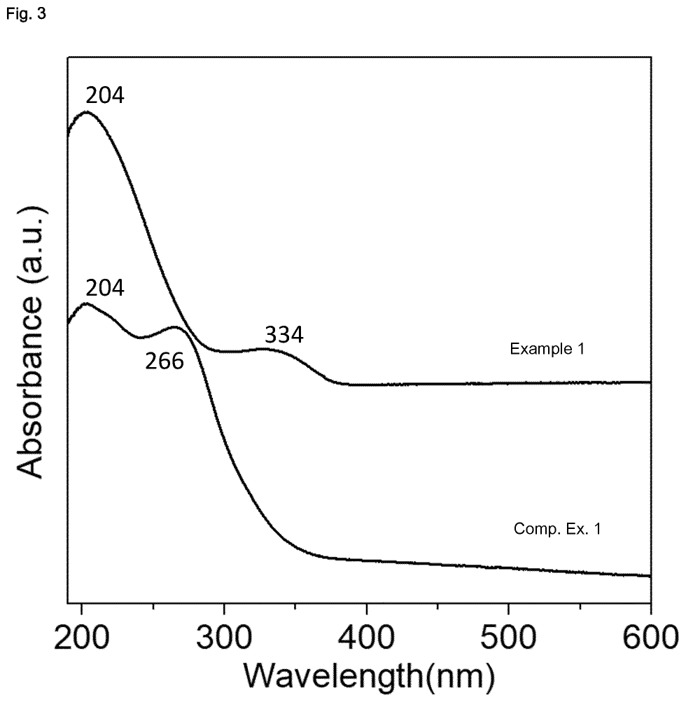

FIG. 4: shows the FT-IR spectra of the zeolitic materials of Example 1 and Comparative Example 1. The wavenumber in cm-1 is shown on the abscissa and the absorbance is shown in arbitrary units on the ordinate. An absorbance band having a maximum wavenumber in the range of from 3740 to 3725 cm-1 can be attributed to “external” and “internal” Si—OH groups whereas an absorbance band having a maximum wavenumber at about 3500 cm-1 can be attributed to a Si—OH “nest”.

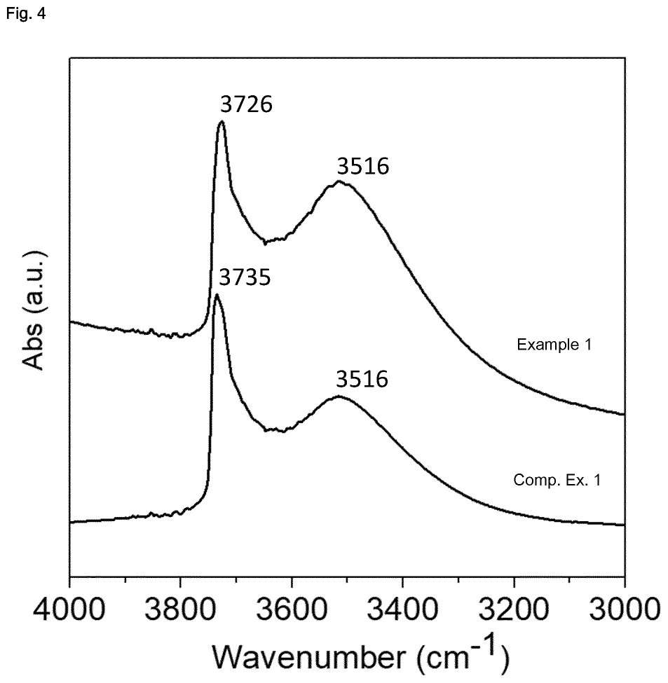

### EXPERIMENTAL SECTION

### Reference Example 1: Determination Methods

### Reference Example 1.1: Determination of XRD Diffraction Spectra

X-ray powder diffraction (XRD) was performed on a Rint-Ultima III (Rigaku) instrument using a Cu Kalpha radiation (Lambda=1.5406 angstrom, 40 kV, 40 mA).

### Reference Example 1.2: Determination of Inductively Coupled Plasma Mass Spectrometry (ICP-MS)

Elemental analyses were performed on an inductively coupled plasma-atomic emission spectrometer (ICP-AES, Shimadzu ICPE-9000). The samples were dissolved in dilute HF solutions.

### Reference Example 1.3: Determination of N2 Adsorption-Desorption Measurements

Nitrogen adsorption-desorption measurements were performed on a BEL-mini analyzer, BEL Japan. Prior to the measurements, all samples were degassed at 350° C. for 2 h.

### Reference Example 1.4: Determination of Water Adsorption

Determination of the water adsorption properties of the examples of the experimental section was performed on a BEL-mini analyzer (BEL Japan) at 25° C. Before a measurement was started, residual moisture of a sample was removed by degassing of the sample at 350° C. for 2 h. The water adsorption of a sample was taken after the sample was exposed to a relative humidity of 91%.

### Reference Example 1.5: Determination of Temperature Programmed Desorption of Ammonia (NH₃-TPD)

Temperature-programmed desorption of ammonia (NH₃-TPD) profiles were recorded on a Multitrack TPD equipment (JapanBEL) using ca. 30 mg of a sample.

### Reference Example 1.6: Determination of NMR Spectra

The high-resolution 29Si MAS NMR spectra were obtained on a JEOL ECA-600 spectrometer.

### Reference Example 1.7: Determination of IR Spectra

FTIR spectra were collected on a JASCO FT-IR 4100 spectrometer. Typically, the H-form samples were pressed into a self-supporting wafer (20 mm diameter, 30±2 mg) and placed in an IR cell, where it was pretreated by evacuation at 723 K for 1 h. After this, the temperature was decreased to 423 K and the spectrum was recorded.

### Reference Example 1.8: Determination of UV-Vis Spectra

The UV-visible diffuse reflectance spectra were recorded on the V-650DS spectrometer (JASCO) using BaSO₄ as a reference.

### Reference Example 1.9: Scanning Electron Microscopy (SEM)

Field-emission scanning electron microscopy (FE-SEM) were obtained on a Hitachi S-5200 microscope.

### Example 1: Preparation of a Zeolitic Material According to the Present Invention

Tetraethyl orthosilicate (TEOS, Wako, 97%) and titanium tetra-n-butoxide (TBOT, Wako, at least 95.0%) were used as Si and Ti sources, respectively. Tetrapropylammonium hydroxide (TPAOH, TCI, 40% in water) was used as organic structure directing agent (OSDA), and deionized water (Wako, 20 L) was used.

In a typical synthesis, 0.119 g TBOT was mixed with 1.43 g TEOS (⅔ of the total amount) and the mixture added to 0.61 g of an aqueous solution comprising 40 weight-% TPAOH and 2.87 g H₂O. The resulting gel mixture had the following molar ratio: 1 Si: 0.0033 Ti: 0.12 TPAOH: 16 H₂O. Next, the gel mixture was put in an oven, stirred at 50° C. for 30 min and then at 80° C. for 2 h for hydrolysis of TEOS and TBOT, whereby the formed alcohol was removed. After this hydrolysis process, 0.72 g TEOS (⅓ of the total amount) was added into the gel mixture and the above described hydrolysis process was repeated again, i.e. the gel mixture was put in the oven, stirred at 50° C. for 30 min and then at 80° C. for 2 h. After said steps of hydrolysis, the gel mixture was hydrothermally treated in a 20 mL autoclave with Teflon-inner at 170° C. for 2 days under tumbling condition (40 rpm). The obtained products were collected and washed with distilled water and dried at 100° C. overnight. The as-made samples were calcined at 550° C. for 6 h in air to remove the OSDA. The water adsorption of the obtained zeolitic material was 14.3 weight-%. Further, characteristics of the obtained zeolitic material are noted in Table 1 in Reference Example 2 below.

### Comparative Example 1: Preparation of a Zeolitic Material not in Accordance with the Present Invention

For the gel preparation, 500 g tetraethylorthosilicate (TEOS) and 15 g tetraethylorthotitanate (TEOTi; Merck) were filled into a beaker. Then, a solution of 300 g de-ionized water and 220 g aqueous tetrapropylammonium hydroxide (TPAOH; 40 weight-% in water) was added under stirring (200 rpm). The resulting mixture had a pH of 13.5. The mixture was hydrolyzed at room temperature for 60 min during which the temperature rose to 60° C. The mixture had a pH of 12.6 then. Afterwards the ethanol was distilled off until the sump reached a temperature of 95° C. 540 g of distillate was obtained from distillation.

The synthesis gel was then cooled to 40° C. under stirring and 542 g de-ionized water added thereto. The resulting mixture had a pH of 11.9.

The synthesis gel was then transferred into an autoclave. The synthesis gel was heated under stirring in the autoclave to a temperature of 175° C. and stirred at said temperature for 16 h under autogenous pressure. The pressure was in the range of from 8.4 to 10.9 bar (abs). The resulting suspension was then worked-up. To this effect, the resulting suspension was diluted with de-ionized water, wherein the weight ratio of the suspension to de-ionized water was 1:1. Then, about 164 g nitric acid (10 weight-% in water) were added and the resulting mixture had a pH of 7.35. The obtained solids were filtered off and washed four times with de-ionized water (each time 1000 ml de-ionized water were used). Subsequently, the solids were dried in an oven in air at 120° C. for 16 h and then calcined in air at 490° C. for 5 h, wherein the heating rate for calcining was 2° C./min.

The thus obtained TS-1 material had a Si content of 43 weight-%, a Ti content of 2 weight-% and a total loss of carbon of less than 0.1 weight-%. The water adsorption was 15.3 weight-%. The crystallinity was 88% as determined by X-ray diffraction.

### Reference Example 2: Characteristics of the Zeolitic Materials According to Example 1 and Comparative Example 1

TABLE 1
Characteristics of the zeolitic materials of Example 1, and Comparative Example 1.

| | | | | | Maxima of abs. | |
|---|---|---|---|---|---|---|
| | | | | Total amout | bands B1 and B2 | |
| | Si/Ti | S_{BET} | V_{micro} | of acid sites | in FT-IR | Intensity ratio of |
| sample | [mol:mol] | [m² g⁻¹] | [cm³ g⁻¹] | [mmol g⁻¹] | [cm⁻¹] | B1:B2 |
| Comp. | 38 | 449 | 0.188 | 0.037 | B2: 3735; | 0.62:0.82 = |
| Ex. 1 | | | | | B1: 3516 | 0.76 |
| Example | 44 | 439 | 0.192 | 0.049 | B2: 3726; | 0.83:0.95 = |
| 1 | | | | | B1: 3516 | 0.87 |

### Example 3: Catalytic Testing-Propylene Epoxidation

Propylene epoxidation was carried out in an autoclave reactor with a 100 mL Teflon-inner. The autoclave reactor was equipped with a water cooling system, an agitator blade, and a pressure gauge ranging from 0.5 to 3 MPa. Typically, 100 mg catalyst, 8.1 mL methanol, 30 mmol H₂O2 (35 weight-%, containing 1.9 mL H₂O) were added into the reactor. Next, propylene was charged into the autoclave at 0.2 MPa for three times to replace the air inside. The propylene pressure inside the autoclave was maintained at 0.4 MPa during the reaction. The reaction mixture was stirred at 120 rpm and heated to 333 K at 8° C./min. After holding at 333 K for 1 h, the stirring and heating of the autoclave were stopped and the Teflon-inner was taken out and cooled down to room temperature rapidly in an ice bath.

Then, the resulting liquid products were separated from the solids by injection filtration, and determined by a gas chromatograph (Shimadzu GC-14B) equipped with an DB-WAX column (60 m, 0.25 mm diameter, 0.25 μm film) and FID detector, using DMF as internal standard. The amount of unconverted H₂O2 was determined by standard titration method with 0.1 M Ce (SO₄)₂ solution.

The results of the catalytic testing are shown in Table 2 below for the zeolitic materials of Example 1 and Comparative Example 1.

TABLE 2
Results of the catalytic testing in the epoxidation of propylene to propylene oxide.

| Sample | n mmol | | | | Yield % | | | | Sel. % | | | | H₂O₂ | |
|---|---|---|---|---|---|---|---|---|---|---|---|---|---|---|
| No. | PO | PG | BP-1 | BP-2 | PO | PG | BP-1 | BP-2 | PO | PG | BP-1 | BP-2 | Conv. | Eff. |
| Comp. | 3.2 | 1.3 | 3.4 | 6.3 | 10.8 | 4.3 | 11.2 | 21.0 | 22.8 | 9.1 | 23.7 | 44.4 | 37 | 128 |
| Ex. 1 | | | | | | | | | | | | | | |
| Ex. 1 | 5.3 | 3.1 | 5.4 | 10.0 | 17.5 | 10.2 | 18.0 | 33.3 | 22.1 | 12.9 | 22.8 | 42.2 | 69 | 114 |

The following formulas were used for calculating the respective data:

$${Y}_{PO} = \frac{{n}_{PO}}{{n}_{H 2 O 2}^{0}}$$

$${Y}_{PG} = \frac{{n}_{PG}}{{n}_{H 2 O 2}^{0}}$$

$${Eff}_{H 2 O 2} = \frac{{n}_{PO} + {n}_{PG} + {n}_{byproduct}}{{Conv}_{H 2 O 2} \cdot {n}_{H 2 O 2}^{0}}$$

$${S}_{PO} = \frac{{n}_{PO}}{{n}_{PO} + {n}_{PG} + {n}_{byproduct}}$$

$${S}_{PG} = \frac{{n}_{PG}}{{n}_{PG} + {n}_{PO} + {n}_{byproduct}}$$

$${Conv}_{H 2 O 2} = \frac{{n}_{H 2 O 2}^{0} - {n}_{H 2 O 2}}{{n}_{H 2 O 2}^{0}}$$

n_{H2O2}^{O} and N_{H2O2} are the amount (mmol) of H₂O₂ before and after reaction, respectively;

n_{PO}, n_{PG}, and n_{byproduct} are the amount (mmol) of propylene oxide, propylene glycol, and byproducts after reaction;
byproducts are designated as BP, wherein BP-1 is 1-methoxy-2-propanol and BP-2 is 2-methoxy-propan-1-ol.

As can be seen from the results, the zeolitic materials in accordance with Example 1 achieved a comparatively higher yield of propylene oxide (PO) and also a higher conversion of H₂O2. In particular, the zeolitic material of Example 1 showed an outstanding yield of propylene oxide.

### CITED PRIOR ART

WO 2011/064191 A1
WO 2021/123227 A1
WO 2020/221683 A1
WO 2020/074586 A1
Xiujuan Deng et al (“Low-Cost Synthesis of Titanium Silicalite-1 (TS-1) with Highly Catalytic Oxidation Performance through a Controlled Hydrolysis Process”, INDUSTRIAL & ENGINEERING CHEMISTRY RESEARCH, vol. 52, no. 3, 23 Jan. 2013 (2013-01-23), pages 1190-1196)
Zhang Jian Hui et al (“Synthesis of nanosized TS-1 zeolites through solid transformation method with unprecedented low usage of tetrapropylammonium hydroxide”, MICROPOROUS AND MESOPOROUS MATERIALS, vol. 217, pages 96-101)

1.-15. (canceled)

16. A process for the production of a zeolitic material, wherein the framework structure of the zeolitic material comprises Si, O, and a tetravalent element M other than Si, wherein M is selected from the group consisting of Ti, Sn, Zr, Ge, and mixtures of two or more thereof, the process comprising:
(1) preparing a mixture comprising one or more sources of MO₂, one or more organotemplates, a first portion of one or more sources of SiO₂, and a protic solvent system comprising water;
(2) heating the mixture obtained in (1) at a temperature comprised in the range of from 30° C. to the boiling point of the mixture;
(3) heating the mixture obtained in (2) under autogenous pressure at a temperature comprised in the range of from 100 to 250° C.; wherein during the course of (2), a second portion of one or more sources of SiO₂ is added to the mixture.

17. The process of claim 16, wherein M is selected from the group consisting of Ti, Sn, and a mixture thereof.

18. The process of claim 16, wherein heating in (2) is conducted continuously or intermittently.

19. The process of claim 18, wherein heating in (2) is conducted intermittently, wherein the heating consists of two or more heating phases, wherein the second portion of one or more sources of SiO₂ is added during the one or more intervals in between the two or more heating phases.

20. The process of claim 18, wherein heating consists of 2 to 6 heating phases.

21. The process of claim 16, wherein the first portion of one or more sources of SiO₂ comprises an amount in the range of from 45 to 90 mol-% of the total amount (100 mol.-%) of the one or more sources of SiO₂, calculated as SiO₂, added to the mixture in (1) and during the course of (2).

22. The process of claim 16, wherein the second portion of one or more sources of SiO₂ comprises an amount in the range of from 10 to 55 mol-% of the total amount (100 mol.-%) of the one or more sources of SiO₂, calculated as SiO₂, added to the mixture in (1) and during the course of (2).

23. The process of claim 16, further comprising one or more of
(4) isolating the zeolitic material obtained in (3);
(5) optionally washing the zeolitic material obtained in (3) or (4);
(6) drying the zeolitic material obtained in (3), (4) or (5);
(7) calcining the zeolitic material obtained in (3), (4), (5) or (6).

24. A zeolitic material obtained by the process according to claim 16.

25. A zeolitic material, wherein the framework structure of the zeolitic material comprises Si, O, and a tetravalent element M other than Si, wherein M is selected from the group consisting of Ti, Sn, Zr, Ge, and mixtures of two or more thereof, and wherein the UV-vis spectrum of the zeolitic material displays a first absorption band A1 having a maximum in the range of from 180 to 230 nm, and a second absorption band A2 having a maximum in the range of from 290 to 370 nm.

26. The zeolitic material of claim 25, wherein the UV-vis spectrum displays no further maximum between the maximum of the first absorption band and the maximum of the second absorption band.

27. The zeolitic material of claim 25, wherein the FTIR spectrum of the zeolitic material displays a first absorption band B1 having a maximum in the range of from 3,450 to 3,550 cm⁻¹,
and a second absorption band B2 having a maximum in the range of from 3,700 to 3,775 cm⁻¹, wherein the intensity ratio B1:B2 of the first absorption band to the second absorption band is comprised in the range of from 0.70:1 to 0.90:1.

28. A process for preparing a molding, comprising:
(A) providing a zeolitic material according to claim 24;
(B) mixing the zeolitic material provided in step (A) with one or more binders;
(C) optionally kneading of the mixture obtained in step (B);
(D) molding of the mixture obtained in step (B) or (C) to obtain one or more moldings;
(E) drying of the one or more moldings obtained in step (D); and
(F) calcining of the dried molding obtained in step (E).

29. A molding obtained by the process of claim 28.

30. A method comprising utilizing a zeolitic material according to claim 24 as a catalyst, catalyst component, absorbent, adsorbent, or for ion exchange.
